(19) United States
(12) Patent Application Publication
(10) Pub. No.: US 2013/0109650 A1
(43) Pub. Date: May 2, 2013

(54) COMPOSITION CONTAINING OLIGOSACCHARIDES FOR THE TREATMENT/PREVENTION OF INFECTIONS

(71) Applicant: N.V. Nutricia, Zoetermeer (NL)

(72) Inventors: Belinda POTAPPEL-VAN 'T LAND, Kootwijk (NL); Johan GARSSEN, Nieuwegein (NL); Gunther BOEHM, Echzell (DE); Bernd STAHL, Rosbach-Rodheim (DE)

(73) Assignee: N.V. Nutricia, Zoetermeer (NL)

(21) Appl. No.: 13/714,883

(22) Filed: Dec. 14, 2012

Related U.S. Application Data
(63) Continuation of application No. 13/350,389, filed on Jan. 13, 2012.
Application No. 13/350,389 is a continuation of application No. 12/096,573, filed on Oct. 29, 2008, now abandoned.

(51) Int. Cl. A61K 31/702

(52) U.S. Cl. CPC A61K 31/702
USPC 514/54

(57) ABSTRACT

The present invention relates to oligosaccharide mixtures and their use in the treatment and/or prevention of infections, and in particular, for reducing the severity of childhood infection or in the treatment and/or prevention of childhood infection.

### BACKGROUND OF THE INVENTION

1. Field of the Invention

The present invention relates to the use of oligosaccharide mixtures for the treatment and/or prevention of infections.

2. Description of the Background Art

Oligosaccharides, particularly galactooligosaccharides and fructopolysaccharides are often included in nutritional composition for their bifidogenic effects. Recently, new activities of specific oligosaccharides have been described.

WO 2005039597 relates to a method for enhancing the immune system and the treatment and/or prevention of immune system related disorders in a mammal, particularly newborns, said method comprising the administration of uronic acid oligosaccharide and neutral oligosaccharide. Food compositions suitable for use in the above method are also provided.

Boehm G et al., Prebiotics in infant formulas: immune modulators during infancy Nutrafoods 2005; 4:51-57 describes that oral administration of GOS/FOS significantly stimulates the cellular (i.e., Th1/Th2) immune balance.

EP 1267891 provides a pharmaceutical or dietetic product, which serves for reducing and/or blocking the adhesion of pathogenic substances and organisms to eukaryotic cells, in particular mammalian cells. The product described contains at least one carbohydrate having an uronic acid unit on one of the ends thereof. Of the, terminal uronic acid units pertaining to the carbohydrates present, 10 to 100% are provided with a double bond that is especially situated between the C₄ and C₅ atom.

### SUMMARY OF THE INVENTION

It has now surprisingly been found that, besides the known action of oligosaccharides, uronic acid oligosaccharides, preferably a mixture of uronic acid and neutral oligosaccharides can reduce the number of systemic viral genome copies in organs, particularly early after infection. Furthermore, it was found that the activity of natural killer cells was also increased after administration of the present oligosaccharides. This strongly suggests that the oligosaccharides stimulate the innate immunity. To date, only the stimulation of the adaptive immunity with oligosaccharides has been described.

The consequences of these findings are that the present oligosaccharides can be advantageously used to reduce the severity, prevent or treat selected childhood infections. Furthermore, the insight enables the use of the present oligosaccharide to reduce the severity of symptoms of viral infections, such as febrile events and fever.

The invention thus relates to the use of uronic acid saccharides, preferably combined with neutral oligosaccharides, for the manufacture of a pharmaceutical or nutritional composition for reducing the severity of childhood infection or the treatment and/or prevention of childhood infection.

In one embodiment the invention relates to the use of uronic acid saccharides, preferably combined with neutral oligosaccharides, for the manufacture of a pharmaceutical or nutritional composition for the treatment and/or prevention a symptom selected from the group consisting of febrile sickness, febrile event, fever and febrile seizures.

### DETAILED DESCRIPTION OF PREFERRED EMBODIMENTS

The present invention provides a method or the treatment and/or prevention of childhood infection, said method comprising the administration a composition comprising uronic acid oligosaccharides. In a preferred embodiment the present invention relates to a method for reducing the severity of childhood infection. The present invention is particularly useful to prevent systemic childhood infections.

The childhood infection is typically an infection which occurs in the first period of life, particularly in the first five years of life. The immune system of the infant is normally insufficiently developed, making the infant or child susceptible to (systemic) viral infection. Preferably the systemic childhood infection is a disease and/or bacterial or viral infection with a pathogen, selected from the group consisting of chickenpox, coxsackie virus, croup, cytomegalovirus, encephalitis, fifth disease, meningitis, mumps, rubella, rubeola, scarlet fever, shigella, tetanus, respiratory syncytial virus, adenovirus and tuberculosis. In a preferred embodiment the present invention relates to a method for reducing the severity of childhood infection.

Because the severity of infection is reduced, the present invention also provides a method for the treatment and/or prevention of an infection symptom selected from the group consisting of febrile sickness, febrile event, fever, febrile seizures, said method comprising the administration a composition comprising uronic acid oligosaccharides. The term febrile sickness also encompasses terminology like the reduced incidence of sickness, less sick, reduced incidence of fever and the like.

Uronic Acid Oligosaccharide

The term uronic acid saccharide as used in the present invention refers to an oligosaccharide wherein at least 50% of the residues are selected from the group consisting of guluronic acid, mannuronic acid, galacturonic acid and glucuronic acid. Preferably the uronic acid saccharide is an uronic acid oligosaccharide with a degree of polymerization (DP) of 2 to 100. In a preferred embodiment the uronic acid saccharide comprises at least 50% galacturonic acid based on total uronic acid residues in the uronic acid oligosaccharide, such uronic acid oligosaccharide is hereinafter referred to as “galacturonic acid oligosaccharide”. More preferably, the present uronic acid oligosaccharide is hydrolyzed pectin, preferably polygalacturonic acid, even more preferably prepared by hydrolysis of apple pectin, citrus pectin and/or sugar beet pectin.

In a preferred embodiment, the uronic acid oligosaccharides of the present invention comprises between 25 and 100 wt. % galacturonic acid oligosaccharides with a DP between 2 and 100 based on total weight of galacturonic acid, more preferably between 50 and 100 wt. %, even more preferably between 75 and 100 wt. %. Preferably the composition comprises between 25 and 100 wt. % galacturonic oligosaccharides with a DP between 2 and 50 based on total weight of galacturonic acid, more preferably between 50 and 100 wt. %, even more preferably between 75 and 100 wt. %.

The galacturonic acid oligosaccharides are preferably prepared by enzymatic digestion of pectin with pectin lyase, pectic lyase, endopolygalacturonase and/or pectinase. The present uronic acid oligosaccharide is preferably obtainable by enzymatic digestion of pectin with pectin lyase, pectic lyase, endopolygalacturonase and/or pectinase.

The uronic acid oligosaccharide may be methoxylated and/or amidated. The uronic acid oligosaccharide is preferably indigestible in the upper human intestinal tract and water-soluble.

In a preferred embodiment, at least one of the terminal hexose units of the uronic acid oligosaccharide has a double bond, which is preferably situated between the C₄ and C₅ position of the terminal hexose unit, i.e., between the carbon atoms in the ring to which R₄ and R₅ are attached. The double bond provides effectively protects against attachment of the pathogenic bacteria to the epithelium. Preferably one of the terminal hexose units comprises the double bond. The double bond at a terminal hexose unit is preferably obtained by enzymatically hydrolyzing pectin with lyase.

Preferably the uronic acid oligosaccharide has the structure I below, wherein the terminal hexose (left) preferably comprises a double bond. The hexose units other than the terminal hexose unit(s) are preferably uronic acid units, preferably galacturonic acid units. The carboxylic acid groups on these units may be free or (partly) esterified, and preferably at least 10% is methylated (see below).

### Structure I

### Polymeric Acid Oligosaccharide

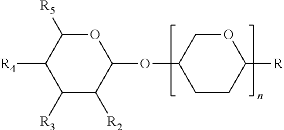

wherein:
R is preferably selected from the group consisting of hydrogen, a hydroxyl group or an acid group, preferably hydroxyl; and
at least one of R₂, R₃, R₄ and R₅ represents N-acetylneuraminic acid, N-glycoloyl-neuraminic acid, free or esterified carboxylic acid, a sulfuric acid group or a phosphoric acid group, and the remaining of R₂, R₃, R₄ and R₅ represent hydroxyl and/or hydrogen. Preferably one of R₂, R₃, R₄ and R₅ represents N-acetylneuraminic acid, N-glycoloylneuraminic acid, free or esterified carboxylic acid, sulfuric acid group or phosphoric acid group, preferably a free or esterified carboxylic acid, and the remaining of R₂, R₃, R₄ and R₅ groups represent hydroxyl and/or hydrogen. Even more preferably, one of R₂, R₃, R₄ and R₅ represents free or esterified carboxylic acid and the remaining of R₂, R₃, R₄ and R₅ represent hydroxyl and/or hydrogen; and
n is an integer that refers to the number of hexose units (see also Degree of Polymerization, DP), which may be any hexose unit. Suitably n is an integer between 1-249, preferably between 1 and 99, more preferably between 1 and 49. Preferably the hexose unit(s) is ore are an uronic acid unit(s).
   Most preferably R, R₂ and R₃ represent hydroxyl, R₄ represents hydrogen, R₅ represents carboxylic acid, n is any number between 1 and 99, preferably between 1 and 50, most preferably between 1 and 10, and the hexose unit is preferably galacturonic acid.

In a further embodiment, a mixture of uronic acid oligosaccharides is used, which have a different DP and/or comprise both unsaturated and saturated terminal hexose units. Preferably at least 5%, more preferably at least 10%, even more preferably at least 25% of the terminal hexuronic units of the uronic acid oligosaccharide are unsaturated hexuronic units, such as described above, e.g., a terminal hexose unit of the uronic acid oligosaccharide with a double bond preferably between the carbons in the C₄ and C₅ positions. As each individual uronic acid oligosaccharide preferably comprises only one unsaturated terminal hexuronic unit, preferably fewer than 50% of the terminal hexuronic units are unsaturated hexuronic units (i.e., they comprises a double bond).

A mixture of uronic acid oligosaccharides preferably comprises between 2 and 50% unsaturated terminal hexuronic units based on the total amount of terminal hexuronic units, preferably between 10 and 40%.

The uronic acid oligosaccharide can be derivatized. In one embodiment the uronic acid oligosaccharides are characterized by a degree of methylation above 20%, preferably above 50% even more preferably above 70%. As used herein, “degree of methylation” (also referred to as “degree of esterification” or “DE”) is intended to mean the extent to which free carboxylic acid groups comprised in the polygalacturonic acid chain have been esterified (e.g., by a methyl group). In another embodiment the uronic acid oligosaccharides have a degree of methylation above 20%, preferably above 50%, even more preferably above 70%.

Concentration of Uronic Acid Oligosaccharides

The present composition is preferably a nutritional composition, comprising fat, digestible carbohydrate and protein. The present nutritional composition preferably comprises between 0.01 and 5 grams uronic acid oligosaccharide with a DP of 2 to 250 per 100 gram dry weight of the nutritional composition, more preferably between 0.05 and 2 grams per 100 gram dry weight. The present nutritional composition preferably comprises between 0.01 and 5 grams galacturonic acid oligosaccharide with a DP of 2 to 250 (preferably a DP of 2-100) per 100 gram dry weight of the nutritional composition, more preferably between 0.05 and 2 grams per 100 gram dry weight.

The present method preferably comprises the administration per day of between 0.05 and 10 grams uronic acid oligosaccharide with a DP of 2 to 100, even more preferably between 0.1 and 5 grams uronic acid oligosaccharides per day.

Neutral Oligosaccharides

In one embodiment, besides uronic acid saccharides, neutral oligosaccharides are used. In other words, the composition for reducing the severity of childhood infection or the treatment and/or prevention of childhood infection or for the treatment and/or prevention a symptom selected from the group consisting of febrile sickness, febrile event, fever and febrile seizures further comprises neutral oligosaccharides. The term “neutral oligosaccharides” as used herein refers to saccharides which have a degree of polymerization (DP) of saccharide units exceeding 2, more preferably exceeding 3, even more preferably exceeding 4, which are not, or are only partially, digested in the intestine by the action of acids or digestive enzymes present in the human upper digestive tract (stomach and small intestine) but which are fermented by human intestinal flora and preferably lack acidic groups. The neutral oligosaccharide is structurally (chemically) different from the uronic acid oligosaccharide.

The term “neutral oligosaccharides” as used herein preferably refers to saccharides which have a degree of polymerization preferably below 60 saccharide units, preferably below 40, even more preferably below 20, most preferably below 10.

The term saccharide units refers to units having a closed ring structure, preferably hexose, e.g., in pyranose or furanose form.

The neutral oligosaccharide preferably comprises at least 90%, more preferably at least 95% saccharide units selected from the group consisting of mannose, arabinose, fructose, fucose, rhamnose, galactose, β-D-galactopyranose, ribose, glucose, xylose and derivatives thereof, based on the total number of saccharide units comprised therein.

In one embodiment, the present invention comprises the administration of a composition which comprises a neutral oligosaccharide selected from the group consisting of galactooligosaccharides, fructopolysaccharides and fructooligosaccharides; and a uronic acid oligosaccharide selected from the group consisting of short chain pectin and short chain alginate.

Short as in short chain pectin and short chain alginate refers to oligosaccharide derived from pectin or alginate with a DP between 2 and 10.

Suitable neutral oligosaccharides are preferably fermented by the gut flora. Preferably the neutral oligosaccharide is selected from the group consisting of:
cellobiose (4-O-β-D-glucopyranosyl-D-glucose), cellodextrins ((4-O-β-D-glucopyranosyl)ₙ-D-glucose), B-cyclodextrins (Cyclic molecules of α-1-4-linked D-glucose; α-cyclodextrin-hexamer, β-cyclodextrin-heptamer, and γ-cyclodextrin-octamer), indigestible dextrin, gentiooligosaccharides (mixture of β-1-6 linked glucose residues, some 1-4 linkages), glucooligosaccharides (mixture of α-D-glucose), isomaltooligo-saccharides (linear α-1-6 linked glucose residues with some 1-4 linkages), isomaltose (6-O-α-D-glucopyranosyl-D-glucose); isomaltriose (6-O-α-D-glucopyranosyl-(1-6)-α-D-glucopyranosyl-D-glucose), panose (6-O-α-D-glucopyranosyl-(1-6)-α-D-glucopyranosyl-(1-4)-D-glucose), leucrose (5-O-α-D-glucopyranosyl-D-fructopyranoside), palatinose or isomaltulose (6-O-α-D-glucopyranosyl-D-fructose), theanderose (O-α-D-glucopyranosyl-(1-6)-O-α-D-glucopyranosyl-(1-2)-B-D-fructofuranoside), D-agatose, D-lyxohexulose, lactosucrose (O-β-D-galactopyranosyl-(1-4)-O-α-D-glucopyranosyl-(1-2)-β-D-fructofuranoside), α-galactooligosaccharides including raffinose, stachyose and other soy oligosaccharides (O-α-D-galactopyranosyl-(1-6)-α-D-glucopyranosyl-β-D-fructofuranoside), β-galactooligosaccharides or transgalacto-oligosaccharides (β-D-galacto-pyranosyl-(1-6)-[β-D-glucopyranosyl]ₙ-(1-4)-α-D glucose), lactulose (4-O-β-D-galacto-pyranosyl-D-fructose), 4′-galatosyllactose (O-D-galactopyranosyl-(1-4)-O-β-D-gluco-pyranosyl-(1-4)-D-glucopyranose), synthetic galactooligosaccharide (neogalactobiose, isogalactobiose, galsucrose, isolactose I, II and III), fructans-Levan-type (β-D-(2→6)-fructofuranosyl)ₙα-D-glucopyranoside), fructans-Inulin-type (β-D-((2→1)-fructo-furanosyl)ₙα-D-glucopyranoside), 1f-β-fructofuranosylnystose (β-D-((2→1)-fructo-furanosyl)ₙB-D-fructofuranoside), xylooligosaccharides (B-D-((1→4)-xylose)ₙ, lafinose, lactosucrose and arabinooligosaccharides.

According to a further preferred embodiment the neutral oligosaccharide is selected from the group consisting of fructopolysaccharides, fructooligosaccharides, indigestible dextrins, gal-actooligosaccharides (including transgalactooligosaccharides), xylooligosaccharides, arabino-oligosaccharides, glucooligosaccharides, mannooligosaccharides, fucooligosaccharides and mixtures thereof. Most preferably the neutral oligosaccharide is selected from the group consisting of fructooligosacchararides and transgalactooligosaccharides. In a preferred embodiment transglactooligosaccharides are used.

Suitable oligosaccharides and their production methods are further described in Laere, K. J. M., Degradation of structurally different non-digestible oligosaccharides by intestinal bacteria: glycosylhydrolases of Bi. adolescentis. PhD-thesis (2000), Wageningen Agricultural University, Wageningen, The Netherlands) the entire contents of which is hereby incorporated by reference.

In the present galactooligosaccharides, preferably at least 50% of the saccharide units are galactose. Transgalactooligosaccharides (TOS) are particularly suitable and are, for example, sold under the trademark Vivinal™ (Borculo Domo Ingredients, Netherlands).

In a further preferred embodiment the present method comprises the administration of 2 chemically distinct neutral oligosaccharides. The administration of uronic acid oligosaccharides combined with two chemically distinct neutral oligosaccharides provides an optimal effect.

Preferably the present method comprises the administration of an
uronic acid oligosaccharides (see above);
galactose based neutral oligosaccharide (>50% of the saccharide units are galactose), preferably selected from the group consisting of galactooligosaccharide and transgalactooligosaccharide; and
fructose based and/or glucose based neutral oligosaccharides (>50% of the saccharide units are fructose and/or glucose, preferably fructose), preferably inulin, fructan and/or fructooligosaccharide.

This composition is particularly suited for administration to infants in the age between 0-1 year.

Preferably the method comprises the administration of two chemically distinct neutral oligosaccharides, said chemically distinct oligosaccharides having a different DP and/or different average DP, preferably different average DP. In another embodiment administering chemically distinct neutral oligosaccharides with different average DP, provides an even more optimal immune-modulating effect. Preferably galactose based neutral oligosaccharide has an average DP between 2 and 10, and fructose and/or glucose based neutral oligosaccharides have an average DP between 10 and 60.

The neutral oligosaccharide is preferably administered in an amount of between 10 mg and 100 gram per day, preferably between 100 mg and 50 grams per day, even more preferably between 0.5 and 20 gram per day.

The acid- and neutral oligosaccharides act synergistically. Preferably the acid and neutral oligosaccharides are administered in a weight ratio of between 0.01:1 and 1:0.01, preferably in a weight ratio of between 0.1:1 and 1:0.1.

Foods

It was found that the uronic acid oligosaccharides, and particularly the mixture of acid and neutral oligosaccharides can be advantageously applied in food, such as baby food, infant formula and clinical nutrition. Such food preferably comprises lipid, protein and carbohydrate and is preferably administered in liquid form. The term “liquid food” as used in the present invention includes dry food (e.g., powders) which are accompanied with instructions as to admix said dry food mixture with a suitable liquid (e.g., water).

Hence, the present invention also relates to a nutritional composition which preferably comprises between 5 and 60 en % lipid, between 5 and 40 en % protein, between 15 and 90 en % carbohydrate and the present uronic acid oligosaccharides, preferably in combination with the neutral oligosaccharides. Preferably the present nutritional composition preferably comprises between 10 and 60 en % lipid, between 5 and 40 en % protein and between 25 and 75 en % carbohydrate (en % is short for energy percentage and represents the relative amount each constituent contributes to the total caloric value of the preparation).

Such food preferably is in liquid form and has a limited viscosity. It was found that the foods comprising the uronic acid oligosaccharides, optionally combined with the neutral oligosaccharides, provides a liquid nutrition with sufficiently low viscosity so it can be applied as, e.g., liquid baby foods and liquid clinical food which can be fed through a teat, tube or a straw, while retaining the low viscosity. In a preferred embodiment, the present composition is orally administered to infants and in one embodiment has a viscosity below 600 mPas, preferably below 250 mPas, more preferably below 50 mPas, most preferably below 25 mPas at a shear rate of 100 s⁻¹ at 20° C. Whenever the term viscosity used in the present document, this refers to the physical parameter which is determined according to the following method:

The viscosity may be determined using a Carri-Med CSL rheometer. The used geometry is of conical shape (6 cm 2 deg acrylic cone) and the gap between plate and geometry is set on 55 μm. A linear continuous ramp shear rate is used from 0 to 150 s⁻¹ in 20 seconds.

Stool irregularities (e.g., hard stools, insufficient stool volume, diarrhea) is a major problem in many babies that suffer from an childhood infection and receive liquid foods. The stool problems may be reduced by administering the present oligosaccharides in a liquid that has an osmolality between 50 and 500 mOsm/kg, more preferably between 100 and 400 mOsm/kg. In view of the above, it is also important that the liquid food does not have an excessive caloric density, however still provides sufficient calories to feed the subject. Hence, the liquid food preferably has a caloric density between 0.1 and 2.5 kcal/ml, even more preferably a caloric density of between 0.5 and 1.5 kcal/ml.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

### Example 1

Effect of Uronic Acid Oligosaccharide and Neutral Oligosaccharide on Cytomegalovirus Infection

To evaluate possible systemic protective effects of immune modulation induced by nutritional intervention, a model for murine cytomegalovirus (MCMV) infection was used. In this model, the effect of a prebiotic oligosaccharide mixture was investigated.

C57BL/6J mice were supplemented orally with a mixture comprising galacto-oligosaccharides, fructo-oligosaccharides and uronic acid oligosaccharides (GOS/FOS/AOS) two weeks prior and during systemic infection with MCMV Immunomodulatory effects were analyzed by, e.g., delayed-type hypersensitivity (DTH) measurement as an in vivo parameter for T-helper 1 type of immunity. In addition, in several organs viral load was measured using a quantitative polymerase chain reaction technique (Q-PCR).

Within mice receiving the prebiotic mixture, MCMV DNA copy numbers were significantly reduced in multiple organs especially early after infection. Furthermore, a MCMV-specific DTH response could be detected in both groups. Yet, the time needed to develop a MCMV-specific DTH response differed significantly between groups. In mice receiving a placebo diet, DTH reached significance at day 6 post infection, whereas in mice receiving GOS/FOS/AOS, the onset of DTH response was delayed and reached significance at day 14. It is suggested that the delay in onset of DTH immunity is due to a lower pathogenic/antigenic load in supplemented mice.

In conclusion, this study suggests that supplementation with a prebiotic oligosaccharide mixture influences early innate immunity and as such affects the systemic MCMV infection in C57BL/6J mice.

The outcome of this study is indicative for the advantageous use of uronic acid saccharides, preferably combined with neutral oligosaccharides for the treatment and/or prevention of childhood infection and/or for the treatment and/or prevention a symptom selected from the group consisting of febrile sickness, febrile event, fever and febrile seizures.

### Example 2

### A Composition for Infant Nutrition

A liquid infant nutrition, prepared by admixing 13.9 g powder with water to yield 100 ml final product, said liquid product comprising per 100 ml:

| | |
|---|---|
| Energy | 66 kcal |
| Protein | 8 en % |
| | 1.3 g (comprising 0.5 g casein; 0.7 g whey; |
| | 0.072 g L-arginine) |
| Digestible | 44 en % |
| Carbohydrates: | 7.4 g (comprising 7.3 g lactose) |
| Fat: | 48 en % |
| | 3.5 g (comprising 0.41 g linoleic acid; 0.08 g |
| | α-linolenic acid; 0.012 g arachidonic acid; 0.002 g |
| | eicosapentanoic acid; 0.006 g docosahexaenoic |
| | acid; 1.4 g oleic acid;) |
| Fiber: | 0.8 g (comprising 0.05 g fructopolysaccharide |
| | (Raftiline HP ™, Orafti, Tienen, Belgium); |
| | 0.55 g transgalactooligosaccharides (Vivinal-GOS ™ |
| | (Borculo Domo Ingredients, Netherlands); 0.20 g |
| | pectin hydrolysate prepared as described in EP |
| | 1373543, Example 1. |
| Osmolarity: | 300 mOsmol/l |

The composition further comprises choline (6 mg/100 ml) and taurine (6.3 mg/100 ml); minerals and trace elements (including 2 mg zinc/100 ml) and vitamins in amounts in compliance with the international guidelines for infant milk formula.

We claim:

1. A use of
(a) a galacturonic acid oligosaccharide prepared by enzymatic digestion of pectin with pectin lyase, pectic lyase, endopolygalacturonase and/or pectinase;
(b) a galactose based neutral oligosaccharide; and
(c) a fructose based neutral oligosaccharide,
for the manufacture of a nutritional composition for oral administration to an infant, said composition comprising
(i) between 5 and 60 en % lipid,
(ii) between 5 and 40 en % protein, and
(iii) between 15 and 90 en % carbohydrate,
for the treatment and/or prevention in said infant of a symptom selected from the group consisting of fever and febrile seizures.

2. The use according to claim 1, wherein the galacturonic acid oligosaccharide has a degree of polymerization of 2 to 100.

3. The use according to claim 1, wherein:
(a) the galactose based neutral oligosaccharide is selected from the group consisting of a galactooligosaccharide and a transgalactooligosaccharide, and
(b) the fructose based and/or glucose based neutral oligosaccharide is selected from the group consisting of inulin, a fructopolysaccharide and a fructooligosaccharide.

4. The use according claim 1 wherein the infant is at an age of between 0 and 1 year.

5. A method for treating or preventing fever or febrile seizures in an infant, comprising administering orally to the infant a nutritional composition that comprises between 5 and 60 en % lipid, between 5 and 40 en % protein, between 15 and 90 en % carbohydrate, and
(a) a galacturonic acid oligosaccharide prepared by enzymatic digestion of pectin with pectin lyase, pectic lyase, endopolygalacturonase and/or pectinase;
(b) a galactose-based neutral oligosaccharide; and
(c) a fructose-based neutral oligosaccharide.

6. The method according to claim 5, wherein the galacturonic acid oligosaccharide has a degree of polymerization of 2 to 100.

7. The method according to claim 5, wherein:
(a) the galactose based neutral oligosaccharide is selected from the group consisting of a galactooligosaccharide and a transgalactooligosaccharide, and
(b) the fructose based and/or glucose based neutral oligosaccharide is selected from the group consisting of inulin, a fructopolysaccharide and a fructooligosaccharide.

8. The method according claim 5, wherein the infant is at an age of between 0 and 1 year.
